(19) United States
(12) Patent Application Publication
(10) Pub. No.: US 2025/0387306 A1
(43) Pub. Date: Dec. 25, 2025

(54) WATER OIL WATER EMULSION CONTAINING HIGH METAL SALT CONCENTRATION

(71) Applicant: Honeywell International Inc., Charlotte, NC (US)

(72) Inventors: Siyue Li, Hong Kong (CN); Yanyan Song, Suzhou (CN); Junjia Fang, Suzhou (CN)

(21) Appl. No.: 19/240,957

(22) Filed: Jun. 17, 2025

Related U.S. Application Data
(60) Provisional application No. 63/663,987, filed on Jun. 25, 2024.

(51) Int. Cl. A61K 8/06; A61K 6/60; A61K 6/70; A61Q 11/00

(52) U.S. Cl. CPC A61K 8/066; A61K 6/60; A61K 6/70; A61Q 11/00; A61K 2800/51

(57) ABSTRACT

Provided is a high metal ion concentration emulsion comprising an oil phase and a water phase. The oil phase includes at least two emulsifiers and an oil. The at least two emulsifiers may include a hydrophobic emulsifier and a hydrophilic emulsifier. The water phase includes stannous fluoride, a chelating agent, citric acid, and water.

### CROSS-REFERENCE TO RELATED APPLICATION

This application claims priority to U.S. Provisional Application No. 63/663,987, filed Jun. 25, 2024, which is herein incorporated by reference in its entirety.

### FIELD

The present disclosure relates generally to a double emulsion with a high metal salt content.

### BACKGROUND

Double emulsions (DEs) allow for efficient encapsulation and delivery of active substances, such as anti-inflammatoires, hormones, and antiseptics. The stability of the DEs can be affected by environmental stresses such as pH, temperature, and ionic strength. Known emulsions with high metal content are extremely unstable due to the charge imbalance at the interface between two phases. Hence, using high metal salt content as the active substance in an emulsion is difficult to form a stable emulsion. For example, stannous fluoride (SnF₂) is an effective functional agent in oral care products. However, high concentrations of SnF₂ may lead to unstable emulsions.

A stable high metal salt emulsion is needed.

### SUMMARY

The present disclosure provides a high metal ion concentration emulsion including an oil phase and a water phase. The oil phase includes at least two emulsifiers and an oil. The water phase includes stannous fluoride, a chelating agent, citric acid, and water.

The present disclosure further provides a dental solution including a water-oil-water (W/O/W) emulsion. The W/O/W emulsion includes at least two emulsifiers, an oil, stannous fluoride, a chelating agent, citric acid, and water.

The present disclosure also provides a method of forming a water-oil-water (W/O/W) emulsion including combining a water phase into an oil phase during constant stirring at 100-1000 rpm for 1-60 min to form the W/O/W emulsion. The oil phase includes at least two emulsifiers and an oil. The water phase includes stannous fluoride, a chelating agent, citric acid, and water.

### BRIEF DESCRIPTION OF THE DRAWINGS

The above-mentioned and other features and advantages of this disclosure, and the manner of obtaining them, will become more apparent, and will be better understood by reference to the following description of the exemplary embodiments taken in conjunction with the accompanying drawings, wherein:

FIG. 1A is a photomicrograph of the W/O/W emulsion of the present disclosure diluted 5-fold; 10-fold; and

FIG. 1B is a photomicrograph of the W/O/W emulsion of FIG. 1A diluted 10-fold.

FIG. 1C is a photomicrograph of the W/O/W emulsion of FIG. 1A diluted

Corresponding reference characters indicate corresponding parts throughout the several views. Although the drawings represent embodiments of various features and components according to the present disclosure, the drawings are not necessarily to scale and certain features may be exaggerated in order to better illustrate and explain the present disclosure. The exemplification set out herein illustrates an embodiment of the disclosure, and such an exemplification is not to be construed as limiting the scope of the disclosure in any manner.

### DETAILED DESCRIPTION

For the purposes of promoting an understanding of the principles of the present disclosure, reference is now made to the embodiments illustrated in the drawings, which are described below. The exemplary embodiments disclosed herein are not intended to be exhaustive or to limit the disclosure to the precise form disclosed in the following detailed description. Rather, these exemplary embodiments were chosen and described so that others skilled in the art may utilize their teachings.

The present disclosure provides a stable high metal salt content double emulsion for high-water content oral care systems such as toothpaste and mouthwash.

### I. High Metal Salt Emulsion Complex Composition

The high metal salt emulsion complex may be a water-oil-water double emulsion, or “W/O/W emulsion.” The W/O/W emulsion of present disclosure may stably comprise a high amount of metal ions from metal salts. Comprising an oil phase and a water phase, the W/O/W emulsion can exist stably without phase layering, and with a high retention of Sn²⁺, indicating that Sn²⁺ is encapsulated in an inner water phase. The W/O/W emulsion of the present disclosure may comprise an amount of metal ions, such as Nat from a chelating agent and/or Sn²⁺ from stannous fluoride, from 5 wt. %, 10 wt. %, 15 wt. % to 20 wt. %, 25 wt. %, or 30 wt. %, or any range using any two of the foregoing values as endpoints, such as 5 wt. % to 30 wt. %, 10 wt. % to 25 wt. %, or 15 wt. % to 20 wt. %, based on the total weight of the emulsion. To stabilize a high level of metal salt ions in a composition, a double emulsion may be formed in a single emulsion step.

The W/O/W emulsion may be directly used in a dental solution such as toothpaste and/or mouthwash. The dental solution may comprise an amount of W/O/W emulsion from 0.05 wt. %, 1 wt. %, 3 wt. % to 5 wt. %, 7 wt. %, or 10 wt. %, or any range using any two of the foregoing values as endpoints, such as 0.05 wt. % to 10 wt. %, 1 wt. % to 7 wt. %, or 3 wt. % to 5 wt. %, based on the total weight of the dental solution.

### A. Oil Phase

The oil phase may comprise at least two emulsifiers and an oil. The oil phase may have a high viscosity and low polarity to aid in the formation of the double emulsion.

i. Emulsifiers

The at least two emulsifiers may comprise a hydrophobic emulsifier and a hydrophilic emulsifier. The oil phase may comprise an amount of emulsifier from 10 wt. %, 25 wt. %, 30 wt. % to 32 wt. %, 35 wt. % or 40 wt. %, or any range using any two of the foregoing values as endpoints, such as 10 wt. % to 40 wt. %, 25 wt. % to 35 wt. %, or 30 wt. % to 32 wt. %, based on the total weight of the oil phase. The W/O/W emulsion may comprise an amount of emulsifier from 1 wt. %, 3 wt. %, 5 wt. % to 7 wt. %, 9 wt. %, or 11 wt. %, or any range using any two of the foregoing values as endpoints, such as 1 wt. % to 11 wt. %, 3 wt. % to 9 wt. %, or 5 wt. % to 7 wt. %, based on the total weight of the W/O/W emulsion.

Suitable hydrophilic emulsifiers may include anionic emulsifiers, nonionic emulsifiers, and natural emulsifiers such as span 20, tween 20, tween 40, tween 60, tween 80, PEG-20, PEG-40, PEG-60, PEG-80, PEG-400, AEO-3, AEO-7, AEO-9, DEA, Eumulgin HPS, or their combinations. The oil phase may comprise an amount of hydrophilic emulsifier from 5 wt. %, 8 wt. %, 10 wt. % to 15 wt. %, 18 wt. %, or 20 wt. %, or any range using any two of the foregoing values as endpoints, such as 5 wt. % to 20 wt. %, 8 wt. % to 18 wt. %, or 10 wt. % to 15 wt. %, based on the total weight of the oil phase. The W/O/W emulsion may comprise an amount of hydrophilic emulsifier from 1 wt. %, 2 wt. %, 3 wt. % to 3.5 wt. %, 4 wt. %, or 5 wt. %, or any range using any two of the foregoing values as endpoints, such as 1 wt. % to 5 wt. %, 2 wt. % to 4 wt. %, or 3 wt. % to 3.5 wt. %, based on the total weight of the W/O/W emulsion.

Suitable hydrophobic emulsifiers may include anionic emulsifiers, nonionic emulsifiers, and natural emulsifiers such as glyceryl monostearate, sodium stearoyl lactate, span 40, span 60, span 80, soy lecithin, cete aryl alcohol, sodium stearate sulfate, cetyl phosphate, polyglycerol-poly-ricinolein acid ester, polyglycerol-laurate, polyglycerol-oleate, and their derivatives and combinations. The oil phase may comprise an amount of hydrophobic emulsifier from 5 wt. %, 8 wt. %, 10 wt. % to 15 wt. %, 18 wt. %, or 20 wt. %, or any range using any two of the foregoing values as endpoints, such as 5 wt. % to 20 wt. %, 8 wt. % to 18 wt. %, or 10 wt. % to 15 wt. %, based on the total weight of the oil phase. The W/O/W emulsion may comprise an amount of hydrophobic emulsifier from 1 wt. %, 2 wt. %, 3 wt. % to 3.5 wt. %, 4 wt. %, or 5 wt. %, or any range using any two of the foregoing values as endpoints, such as 1 wt. % to 5 wt. %, 2 wt. % to 4 wt. %, or 3 wt. % to 3.5 wt. %, based on the total weight of the W/O/W emulsion.

ii. Oil

The oil phase may comprise an oil. Suitable oils may include flavor oil, such as a-terpineol, eugenol, limonene, citral, menthol, geraniol, linalyl acetate, vitamin E and related derivatives, and vitamin C and related derivatives. Examples of suitable long chain triglyceride-containing oils include almond oil, babassu oil, borage oil, black currant seed oil, canola oil, castor oil, corn oil, cottonseed oil, olive oil, palm oil, peanut oil, safflower oil, sesame oil, linseed oil, grape oil, coconut oil, soybean oil, cocoa butter, vitamin E oil and other suitable essential oils or their combinations.

The oil phase may comprise an amount of oil from 60 wt. %, 65 wt. %, 70 wt. % to 75 wt. %, 80 wt. %, or 90 wt. %, or any range using any two of the foregoing values as endpoints, such as 60 wt. % to 90 wt. %, 65 wt. % to 80 wt. %, or 70 wt. % to 75 wt. %, based on the total weight of the oil phase. The W/O/W emulsion may comprise an amount of oil from 10 wt. %, 15 wt. %, 20 wt. % to 25 wt. %, 27 wt. %, or 30 wt. %, or any range using any two of the foregoing values as endpoints, such as 10 wt. % to 30 wt. %, 15 wt. % to 27 wt. %, or 20 wt. % to 25 wt. %, based on the total weight of the W/O/W emulsion.

### B. Water Phase

The water phase may comprise any combination of stannous fluoride, a chelating agent, citric acid, and water.

i. Stannous Fluoride

The water phase may comprise an amount of stannous fluoride from 5 wt. %, 7 wt. %, 9 wt. % to 11 wt. %, 13 wt. %, or 15 wt. %, or any range using any two of the foregoing values as endpoints, such as 5 wt. % to 15 wt. %, 7 wt. % to 13 wt. %, or 9 wt. % to 11 wt. %, based on the total weight of the water phase.

The W/O/W emulsion may comprise an amount of stannous fluoride from 3 wt. %, 5 wt. %, 7 wt. % to 9 wt. %, 10 wt. %, or 12 wt. %, or any range using any two of the foregoing values as endpoints, such as 3 wt. % to 12 wt. %, 5 wt. % to 10 wt. %, or 7 wt. % to 9 wt. %, based on the total weight of the W/O/W emulsion.

ii. Chelating Agent

The water phase may comprise a chelating agent. Chelating agents are added to stabilize the tin ions and slow down the oxidation rate in order to maintain the efficacy of stannous ions. Suitable chelating agents may comprise glutamic acid diacetate (GLDA-4Na), aspartic acid N, N diacetic acid (ASDA), GLDA/ASDA salts, and combinations thereof.

The water phase may comprise an amount of chelating agent from 15 wt. %, 21 wt. %, 27 wt. % to 33 wt. %, 39 wt. %, or 45 wt. %, or any range using any two of the foregoing values as endpoints, such as 15 wt. % to 45 wt. %, 21 wt. % to 39 wt. %, or 27 wt. % to 33 wt. %, based on the total weight of the water phase.

The W/O/W emulsion may comprise an amount of chelating agent from 10 wt. %, 15 wt. %, 20 wt. % to 22 wt. %, 25 wt. %, or 30 wt. %, or any range using any two of the foregoing values as endpoints, such as 10 wt. % to 30 wt. %, 15 wt. % to 25 wt. %, or 20 wt. % to 22 wt. %, based on the total weight of the W/O/W emulsion.

iii. Citric Acid

The water phase may comprise an amount of citric acid from 1 wt. %, 1.5 wt. %, 2 wt. % to 2.5 wt. %, 3 wt. %, or 4 wt. %, or any range using any two of the foregoing values as endpoints, such as 1 wt. % to 4 wt. %, 1.5 wt. % to 3 wt. %, or 2 wt. % to 2.5 wt. %, based on the total weight of the water phase.

The W/O/W emulsion may comprise an amount of citric acid from 0.5 wt. %, 1 wt. %, 1.5 wt. % to 2 wt. %, 2.5 wt. %, or 3 wt. %, or any range using any two of the foregoing values as endpoints, such as 0.5 wt. % to 3 wt. %, 1 wt. % to 2.5 wt. %, or 1.5 wt. % to 2 wt. %, based on the total weight of the W/O/W emulsion.

iv. Water

The water phase may comprise an amount of water from 36 wt. %, 55 wt. %, 60 wt. % to 65 wt. %, 70 wt. %, or 79 wt. %, or any range using any two of the foregoing values as endpoints, such as 36 wt. % to 79 wt. %, 55 wt. % to 70 wt. %, or 60 wt. % to 65 wt. %, based on the total weight of the water phase.

The W/O/W emulsion may comprise an amount of water from 15 wt. %, 40 wt. %, 45 wt. % to 50 wt. %, 52 wt. %, or 75 wt. %, or any range using any two of the foregoing values as endpoints, such as 15 wt. % to 75 wt. %, 40 wt. % to 52 wt. %, or 45 wt. % to 50 wt. %, based on the total weight of the W/O/W emulsion.

### II. Method of Preparing High Metal Salt Emulsion—One-step Emulsification

The W/O/W emulsion may be formed from a one-step emulsification. One-step emulsification comprises using a single mixing step in which the water phase is continuously added into the oil phase. The at least two emulsifiers may cause a catastrophic inversion under continuous agitation such that the added water phase is dispersed as droplets and the droplet population increases until phase inversion occurs.

The water phase may be added continuously to the oil phase, comprising the hydrophobic and hydrophilic emulsifiers. Initially, both emulsifiers may be dissolved in the oil to create the oil phase. The oil phase and the water phase may be heated separately in a water bath to a temperature from 55° C., 60° C., 65° C. to 70° C., 75° C., or 80° C., or any range using any two of the foregoing values as endpoints, such as 55° C. to 80° C., 60° C. to 75° C., or 65° C. to 70° C.

The warmed water phase may be slowly added to the warmed oil phase under stirring. The water phase may be stirred into the oil phase at a speed of 100 rpm, 200 rpm, 400 rpm or 600 rpm, 800 rpm, or 1000 rpm, or any range using any of the foregoing values as endpoints, such as 100 rpm to 1000 rpm, 200 rpm to 800 rpm, or 400 rpm to 600 rpm. Further, the water phase may be stirred into the oil phase over a time period from 1 min, 5 min, 10 min to 15 min, 20 min or 30 min, or any range using any of the foregoing values as endpoints, such as 1 min to 30 min, 5 min to 20 min, or 10 min to 15 min.

The W/O/W emulsion mixture may be cooled at room temperature (20-25° C.) with natural slow cooling under stirring at a rate described above for a period of time from 15 min, 30 min, 45 min to 1 hour, 1.5 hours, or 2 hours, or any range using any of the foregoing values as endpoints, such as 15 min to 2 hours, 30 min to 1.5 hours, or 45 min to 1 hour. By controlling the temperature, speed, reaction time, and other parameters of emulsification process, the stable W/O/W emulsion is obtained via phase inversion.

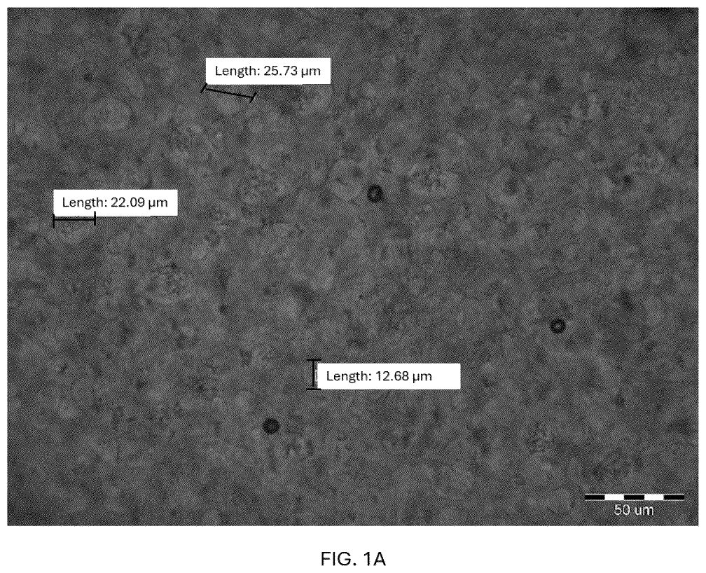

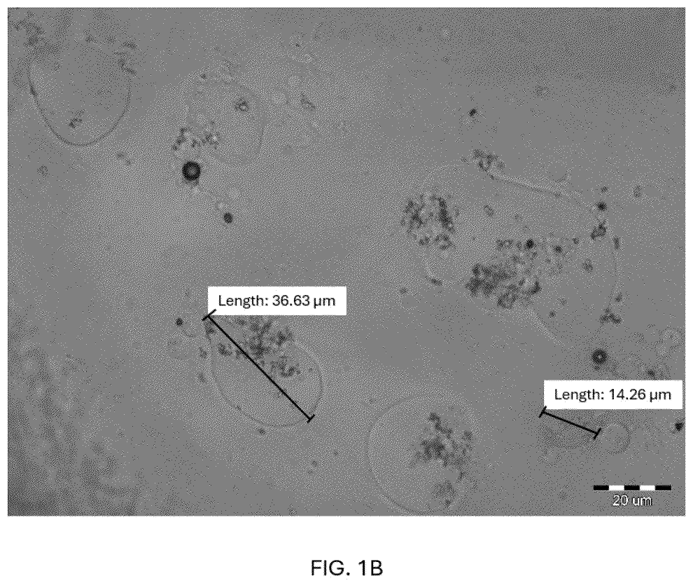

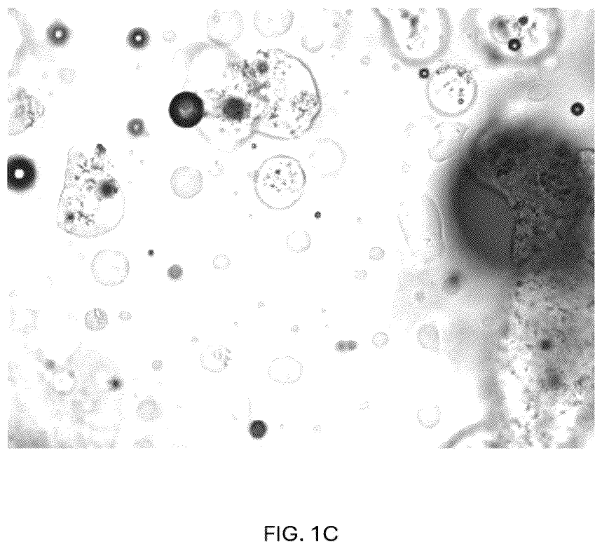

Further, FIG. 1A shows a photomicrograph of the W/O/W emulsion comprising high metal ion concentration diluted 5-fold with glycerol. FIGS. 1B and 1C show the W/O/W emulsion diluted 10-fold with glycerol.

### III. Properties of High Metal Salt Emulsion

### A. Determining the Layers of the W/O/W Emulsion

The conductivity of multiple W/O/W samples were measured (e.g., at 25° C.) using a Themo Scientific Orion VSTAR20 to determine the nature of the external phase of the W/O/W emulsion. Specifically, the multiple emulsions have an outer water layer phase and inner water layer phase where water may be more likely to migrate from the outer into the inner water phase or vice versa. Conductivity may be used to understand the nature of the external phase, as the oil likely does not indicate any electric conductivity.

The electrical conductivity of the W/O/W emulsion may be from 0.1 us/cm, 1 us/cm, or 10 us/cm to 100 us/cm, 500 us/cm, or 1000 us/cm, or any range using any two of the foregoing values as endpoints, such as from 0.1 us/cm to 1000 us/cm, 1 us/cm to 500 us/cm, or 10 us/cm to 100 us/cm, as determined at 25° C. using a Themo Scientific Orion VSTAR20.

As shown in Table A below, the conductivity of the outer layer of the W/O/W emulsion is not substantially 0, indicating that the outer layer is more inclined towards the aqueous/water phase. That is indicative that the one-step multiple emulsion (W/O/W) is formed.

TABLE A
Conductivity of Phases, Emulsion, and Solutions

| Component | Electrical Conductivity | |
|---|---|---|
| Water | 0.63 | us/cm |
| Oil Phase | 0 | us/cm |
| W/O/W Emulsion | 25.0 | us/cm |
| SnF₂ Solution | 15730 | us/cm |
| Demulsified Solution | 1077 | us/cm |

### B. Particle Size

The emulsion was dispersed in water and the particle size of the emulsion was measured (e.g., utzing a Microtrac-s3500). The W/O/W emulsion may have a D50 particle size from 6 μm, 10 μm, 20 μm to 30 μm, 40 μm, 50 μm, or any range using any of the foregoing values as endpoints, such as 6 μm to 50 μm, 10 μm or 40 μm, or 20 μm to 30 μm, as determined by dynamic light scattering ISO 13320-1. The W/O/W emulsion may have a D99.9 particle size from 60 μm, 80 μm, 100 μm to 150 μm, 160 μm, 200 μm, or any range using any of the foregoing values as endpoints, such as 60 μm to 200 μm, 80 μm or 160 μm, or 100 μm to 150 μm, as determined by dynamic light scattering ISO 13320-1.

### C. Stability

The retention of tin ions is used to determine the stability of the stannous ion complex in the solution. Specifically, to test the stability of the stannous ion emulsion complex in solution, an iodine titration for retention testing of tin ions is performed.

Specifically, a Mettler Toledo T50 potentiometric titrator and a platinum electrode may be used as the iodine titration method used to evaluate Sn²⁺ content may use. Here, Sodium thiosulfate solution was used as a reductive agent and iodine solution was used as an oxidized agent. The stannous ion emulsion complex may then be placed in an oven at 40° C. and 75% humidity for a speed up aging process to evaluate the stability. If the stannous ion emulsion complex has a Sn²⁺ retention above 90%, the emulsion complex has a good stability.

TABLE B
Range of Stability of Stannous Ion Emulsion Complex

| | Storage | | | | SnF₂ % |
|---|---|---|---|---|---|
| | Condition | 1 Day | 14 Days | 30 Days | Retention |
| W/O/W | Room | 7.65 | 7.52 | 7.21 | 94.25% |
| emulsion | temperature | | | | |
| | (20-25° C.) | | | | |
| | 40° C. | 7.65 | 7.54 | 7.15 | 93.46% |

In solution, such as a dental solution, the W/O/W emulsion may have a SnF₂ retention greater than 70%, greater than 75%, greater than 80%, greater than 85%, greater than 90%, greater than 95%, or any range using any of the foregoing values as endpoints, such as 70% to 95%, 75% to 90%, or 80% to 85%.

### D. pH

Acidic pH level may lead to demineralization of teeth, damaging enamel. Specifically, teeth demineralize may occur at about a pH of 5.5. While stannous ions are more stable in acidic solution, lower (e.g., acidic) pH can contribute such issues. The W/O/W emulsion of the present disclosure may have a relatively high pH, such as a pH of 5.5, 6, 7 to 8, 9, or 10, or any range using any of the foregoing values as endpoints, such as 5.5 to 10, 6 to 9, or 7 to 8.

The W/O/W stannous ion in an oral care product may have antibacterial efficacy of at least 90%, at least 92%, at least 94%, at least 96%, at least 98%, or at least 99%, as determined by QBT 2738-2023 antibacterial test.

### E. Viscosity

The W/O/W emulsion may have a viscosity from 1 Pa's, 10 Pas, 50 Pas, 100 Pas to 250 Pas, 500 Pas, 750 Pas, or 1000 Pa's, or any range using any of the foregoing values as endpoints, such as 1 Pa's to 1000 Pas, 10 Pa's to 750 Pas, 50 Pa's to 500 Pas, or 100 Pa's to 250 Pa's, as determined according to ASTM D3236.

### EXAMPLES

### Example 1: Formulation of W/O/W Emulsion

The oil phase and water phase were prepared according to Table 1A and 1B, respectively.

TABLE 1A
Formulation of Oil Phase

| | Component | Wt. % |
|---|---|---|
| Oil Phase | Hydrophilic Emulsifier | 14.3 |
| | Hydrophobic Emulsifier | 14.3 |
| | Oil | 71.4 |

TABLE 1B
Formulation of Water Phase

| | Component | Wt. % |
|---|---|---|
| Water Phase | Stannous Fluoride | 10 |
| | Chelating Agent | 25 |
| | Citric Acid | 2 |
| | Water | 63 |

The W/O/W emulsion was prepared according to the method provided above and the formulation listed in Table 2.

TABLE 2
W/O/W Emulsion Formulation

| Component | Wt. % |
|---|---|
| Stannous Fluoride | 7.4 |
| Chelating Agent | 18.5 |
| Citric Acid | 1.5 |
| Oil | 18.5 |
| Hydrophobic Emulsifier | 3.7 |
| Hydrophilic Emulsifier | 3.7 |
| Water | 46.7 |

### Example 2: Formulation of Comparative Formulations 1-3

Comparative (comp.) formulations 1-3 were prepared using one-step emulsification, described above. The comparative formulations were prepared according to the formulations in Table 3.

TABLE 3
Comparative Emulsions 1-3 Formulations

| Composition | Component | Wt. % |
|---|---|---|
| Comp. Emulsion 1 | Stannous Fluoride | 10 |
| | Chelating Agent | 25 |
| | Citric Acid | 2 |
| | Sodium gluconate | 5 |
| | Water | 58 |
| Comp. Emulsion 2 | Stannous Fluoride | 10 |
| | Chelating Agent | 25 |
| | Citric Acid | 2 |
| | Water | 63 |
| Comp. Emulsion 3 | Stannous Fluoride | 10 |
| | Water | 90 |

### Example 3: Formulation of Dental Solutions Comprising W/O/W Emulsion and Comparative Formulations 1-3

The W/O/W emulsion and comparative (comp.) emulsions 1-3 were added to a dental solution (toothpaste) according to Table 4 below.

TABLE 4
Toothpaste Formula Including W/O/W Emulsion
and Comparative Formulations 1-3

| Formula | Component | Wt. % |
|---|---|---|
| W/O/W Emulsion | Silica | 20 |
| Toothpaste 1 | Glycerol | 6 |
| | Sorbitol | 38 |
| | W/O/W emulsion (Table 2) | 6.1 |
| | Xanthan gum | 2.8 |
| | Saccharin sodium | 0.1 |
| | K12 | 2 |
| | Sodium citrate | 1 |
| | Essence | 1 |
| | Water | 23 |
| Comp. 1 | Silica | 20 |
| Toothpaste | Glycerol | 6 |
| | Sorbitol | 38 |
| | Comp. 1 Emulsion (SnF₂ | 4.5 |
| | solution) | |
| | Xanthan gum | 2.8 |
| | Saccharin sodium | 0.1 |
| | K12 | 2 |
| | Sodium citrate | 1 |
| | Essence | 1 |
| | Water | 24.6 |
| Comp. 2 | Silica | 20 |
| Toothpaste | Glycerol | 6 |
| | Sorbitol | 38 |
| | Comp. 2 Emulsion (SnF₂ | 4.5 |
| | solution) | |
| | Xanthan gum | 2.8 |
| | Saccharin sodium | 0.1 |
| | K12 | 2 |
| | Sodium citrate | 1 |
| | Essence | 1 |
| | Water | 24.6 |
| Comp. 3 | Silica | 20 |
| Toothpaste | Glycerol | 6 |
| | Sorbitol | 38 |
| | Comp. 3 Emulsion (SnF₂ | 4.5 |
| | solution) | |
| | Xanthan gum | 2.8 |
| | Saccharin sodium | 0.1 |
| | K12 | 2 |
| | Sodium citrate | 1 |
| | Essence | 1 |
| | Water | 24.6 |

### Example 4: Properties of W/O/W Emulsion and Comparative Formulations 1-3 in Solution

The metal salt (metal ion) retention of the toothpastes from Example 3 was monitored during a speed-up aging test in a 50° C. oven for 1 month. The results are shown in Table 5.

TABLE 5
Stability Test of Toothpaste Formulations

| | Original Status | | Aging (50° C. for 1 month) | | |
|---|---|---|---|---|---|
| Formulation | pH | SnF₂ % | pH | SnF₂ % | Retention |
| W/O/W | 7.05 | 0.4116 | 6.24 | 0.3340 | 81% |
| Emulsion | | | | | |
| Toothpaste 1 | | | | | |
| Comp. 1 | 7.22 | 0.4170 | 6.00 | 0.3111 | 75% |
| Toothpaste | | | | | |
| Comp. 2 | 7.12 | 0.4350 | 6.22 | 0.2795 | 64% |
| Toothpaste | | | | | |
| Comp. 3 | 7.31 | 0.3370 | 5.54 | 0.1916 | 57% |
| Toothpaste | | | | | |

The results showed the toothpaste comprising the W/O/W emulsion toothpaste 1 had the best Sn²⁺ retention compared to simply adding SnF₂, which indicated encapsulated SnF₂ by emulsion having good stability in toothpaste and other oral care products.

TABLE 6
Efficacy Test Results on W/O/W Emulsion Formulation

| Sample | Sn²⁺ wt. % | Condition | Type of Bacteria | Efficacy |
|---|---|---|---|---|
| W/O/W | 7.4 | Contact | Streptococcus | >99.9% |
| emulsion | | 30 s | mutans | |

The above test results showed that W/O/W emulsion have good antibacterial efficacy for Streptococcus mutans.

### Example 5: Properties of W/O/W Emulsion and Comparative Formulations 1-3 in Solution

Formulations for toothpastes comprising the W/O/W emulsion of Table 2 (W/O/W emulsion toothpastes 2-4) is provided in Table 7 below. The metal salt (metal ion) retention of the W/O/W emulsion toothpastes 2-4 were monitored during a sped-up aging test in a 50° C. oven for 1 month. The results are shown in Table 8.

TABLE 7
Toothpaste Formula Including W/O/W Emulsion
and Comparative Formulations

| | W/O/W | W/O/W | W/O/W |
|---|---|---|---|
| | emulsion | emulsion | emulsion |
| | toothpaste 2 | toothpaste 3 | toothpaste 4 |
| Component | Wt. % | Wt. % | Wt. % |
| Silica | 20 | 20 | 20 |
| Glycerol | 6 | 0 | 6 |
| PEG-400 | 0 | 6 | 0 |
| Sorbitol | 38 | 38 | 38 |
| W/O/W emulsion | 6.1 | 6.1 | 6.1 |
| (Table 2) | | | |
| Cellulose gum | 0 | 0.3 | 0.3 |
| Xanthan gum | 2 | 0.7 | 0.7 |
| Sucralose | 0.12 | 0.12 | 0.12 |
| K12 | 2 | 2 | 2 |
| Sodium | 0 | 0.5 | 0 |
| pyrophosphate | | | |
| Sodium citrate | 0.66 | 0 | 0 |
| Citric acid | 0 | 0 | 0.3 |
| Titanium dioxide | 0.5 | 0.5 | 0.5 |
| Essence | 1 | 1 | 1 |
| Water | 24.62 | 24.78 | 24.98 |

TABLE 8
Stability Test of Toothpaste Formulations

| | Original Status | | Aging (50° C. for 1 month) | | |
|---|---|---|---|---|---|
| Formulation | pH | Sn²⁺ % | pH | Sn²⁺ % | Retention |
| W/O/W | 7.17 | 0.3267 | 6.85 | 0.2776 | 85% |
| emulsion | | | | | |
| toothpaste 2 | | | | | |
| W/O/W | 7.01 | 0.2988 | 6.10 | 0.2332 | 78% |
| emulsion | | | | | |
| toothpaste 3 | | | | | |
| W/O/W | 5.70 | 0.2887 | 5.52 | 0.2681 | 93% |
| emulsion | | | | | |
| toothpaste 4 | | | | | |

The results showed that all three formulations of toothpaste containing W/O/W showed better ionic stability after aging. The stability of the ions is better at low pH. Although toothpaste B was stable, the paste turns yellow after aging test. W/O/W emulsion toothpaste 2 was a relatively good formula.

### Example 6: Formulation of Dental Solutions Comprising W/O/W Emulsion and Comparative Commercial Products

W/O/W Emulsion Toothpaste 1, as formulated in Table 4, was compared to a commercial toothpaste (Comparative Toothpaste 4), Nourish teeth at full age (Adult version 01) from P&G Crest (Batch number: 22100386FA) comprising stannous fluoride.

The metal salt (metal ion) retention of W/O/W Emulsion Toothpaste 1 and the Comparative Toothpaste 4 were monitored during a speed-up aging test in a 50° C. oven for 1 month. The results are shown in Table 9.

TABLE 9
Stability Test of Toothpaste Formulations

| | Original Status | | Aging (50° C. for 1 month) | | |
|---|---|---|---|---|---|
| Formulation | pH | Sn²⁺ % | pH | Sn²⁺ % | Retention |
| W/O/W | 7.05 | 0.4116 | 6.24 | 0.334 | 81% |
| Emulsion | | | | | |
| Toothpaste 5 | | | | | |
| Comparative | 6.30 | 0.2852 | 5.80 | 0.2285 | 80% |
| Toothpaste 4 | | | | | |

The results showed that the W/O/W Emulsion Toothpaste 1 stability of metal ions after aging test can be maintained at more than 80%, which can reach the standard of commercial products, such as Comparative Toothpaste 4.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practices in the art to which this disclosure pertains.

What is claimed is:

1. A high metal ion concentration emulsion, comprising:
an oil phase, the oil phase comprising:
at least two emulsifiers; and
an oil; and
a water phase, the water phase comprising:
stannous fluoride;
a chelating agent;
citric acid; and
water.

2. The emulsion of claim 1, wherein the oil phase further comprises:
10-40 wt. % of the at least two emulsifiers; and
60-90 wt. % of the oil, based on the total weight of the oil phase.

3. The emulsion of either claim 1, wherein the water phase further comprises:
5-15 wt. % of the stannous fluoride;
15-45 wt. of the chelating agent;
1-4 wt. % of the citric acid; and
36-79 wt. % of the water, based on the total weight of the water phase.

4. The emulsion of claim 1, further comprising:
2-10 wt. % of the at least two emulsifiers;
10-30 wt. % of the oil;
3-12 wt. % of the stannous fluoride;
10-30 wt. % of the chelating agent;
0.5-3 wt. % of the citric acid;
15-75 wt. % of the water, based on the total weight of the emulsion.

5. The emulsion of claim 1, wherein the at least two emulsifiers comprise a hydrophobic emulsifier and a hydrophilic emulsifier.

6. The emulsion of claim 5, wherein the oil phase comprises:
5-20 wt. % of the hydrophobic emulsifier; and
5-20 wt. % of the hydrophilic emulsifier, based on the total weight of the oil phase.

7. The emulsion of claim 5, further comprising
1-5 wt. % of the hydrophobic emulsifier; and
1-5 wt. % of the hydrophilic emulsifier, based on the total weight of the emulsion.

8. The emulsion of claim 1, wherein the conductivity of the emulsion is in the range of 0.1 to 1000 us/cm.

9. The emulsion of claim 1, wherein the average particle size (D50) of the emulsion is 1-30 μm.

10. The emulsion of claim 1, wherein the viscosity of the emulsion is 1-1000 Pa·s.

11. The emulsion of claim 1, wherein the retention of SnF₂ in the emulsion after being in an oven at 40° C. for 1 month is greater than 90%.

12. The emulsion of claim 1, further comprising 1-20 wt. % of metal ions, based on the total weight of the emulsion.

13. A dental solution, comprising:
a water-oil-water (W/O/W) emulsion, the W/O/W emulsion comprising:
at least two emulsifiers;
an oil;
stannous fluoride;
a chelating agent;
citric acid; and
water.

14. The dental solution of claim 13, further comprising 0.05-10 wt. % of the W/O/W emulsion, based on the total weight of the dental solution.

15. The dental solution of claim 13, wherein the pH of the W/O/W emulsion is 5.5-10.

16. The dental solution of claim 13, wherein the percent retention of SnF₂ after aging for 1 month at 50° C. is greater than 80%.

17. The dental solution of claim 13, wherein the W/O/W emulsion further comprises:
2-10 wt. % of the at least two emulsifiers;
10-30 wt. % of the oil;
3-12 wt. % of the stannous fluoride;
10-30 wt. % of the chelating agent;
0.5-3 wt. % of the citric acid;
15-75 wt. % of the water, based on the total weight of the emulsion.

18. The dental solution of claim 13, wherein the at least two emulsifiers comprises a hydrophobic emulsifier and a hydrophilic emulsifier.

19. A method of forming a water-oil-water (W/O/W) emulsion, comprising:
combining a water phase into an oil phase during constant stirring at 100-1000 rpm for 1-60 min to form the W/O/W emulsion;
wherein the oil phase comprises:
at least two emulsifiers; and
an oil; and
wherein the water phase comprises:
stannous fluoride;
a chelating agent;
citric acid; and
water.

20. The method of claim 19, further comprising:
heating the oil phase and the water phase to 55-80° C. before combining; and
comprising cooling the W/O/W emulsion at room temperature under stirring at 100-1000 rpm for 15 min to 2 hours.
